(19) United States
(12) Patent Application Publication
(10) Pub. No.: US 2025/0090760 A1
(43) Pub. Date: Mar. 20, 2025

(54) END OF DOSE INDICATORS

(71) Applicant: Owen Mumford Limited, Woodstock (GB)

(72) Inventor: David Robert Blount, Woodstock (GB)

(21) Appl. No.: 18/883,155

(22) Filed: Sep. 12, 2024

(30) Foreign Application Priority Data
Sep. 15, 2023 (GB) 2314121.1

(51) Int. Cl. A61M 5/315

(52) U.S. Cl. CPC A61M 5/31533

(57) ABSTRACT

An injector for use with a primary package. The injector comprises lower and upper housing parts in telescopic engagement for receiving the primary package. A movable shroud is coupled to the lower housing part. An indicating component is releasably coupled to the shroud and a biasing member biases the indicating component away from the shroud. The shroud and indicating component are configured to move together in a rearward direction through the upper housing part during telescopic collapsing of the lower and upper housing parts up to a release position at which position the indicating component is released from the shroud allowing the biasing means to drive axial separation of the indicating component and the shroud, causing the indicating component to continue rearward movement up to a stop position whilst biasing the shroud towards an extended position to cover a cannula tip.

### TECHNICAL FIELD

The present invention relates to end of dose indicators for injectors.

### BACKGROUND

PCT/GB2019/050317 describes a so-called “primary package” comprising a cartridge or syringe for containing a medicament or “drug” in combination with a cannula component. This primary package 1 is illustrated in FIG. 1, where the cartridge is identified by reference numeral 2 and the cannula component by reference numeral 3. Considering each component in more detail, the cartridge comprises a generally cylindrical glass or plastics body 4 having a moveable bung 5 located at an end distal from the end of the body to be connected to the cannula component. The other end of the body 4 is shaped for connection to the cannula component 3 and is closed by a cap 6 comprising a sealing element 7 in the form of a disc shaped elastomeric septum. The cannula component 3 comprises a cannula 8 glued into a hub 9 which is in turn slidably located within a generally cylindrical body 9a. The cannula is enclosed within a rigid needle shield or boot 10 which engages with the hub 9. The boot has an opening therein that is closed by an antibacterial barrier 11. The barrier 11 is permeable to air to prevent external pressure fluctuations from damaging the apparatus.

The cartridge is typically filled with a drug in a sterile environment, typically prior to sealing by the septum. Within this same sterile environment, the cap 6 with antimicrobial barrier is then secured to the body 4, after which the cannula component 3 is attached. In this state, the end of the cannula 8 is held off of the sealing element 7 so that there is no fluid communication between the two components 2, 3.

PCT/EP2022/054940 describes a manual injector that provides for the manual injection of a dose from the primary package 1. The manual injector comprises a lower housing part 22 shaped to receive and accommodate the primary package 1. The manual injector further comprises an upper housing part 32 within which a plunger is concentrically located. During assembly of the manual injector, the primary package is inserted into the lower housing part and the upper housing part is positioned over and around an open end of the lower housing part. In use, a user grasps the upper housing part within his or her fist, and presses the lower end of the manual injector against the injection site. Telescoping of the upper housing part towards the lower housing part causes the plunger to advance into the lower housing part and push against the bung of the primary package. The primary package is advanced through the lower housing part, causing the tip of the cannula to exit through an opening of the lower housing part and penetrate the skin at the injection site. Further force applied by the user advances the plunger and bung through the cartridge body of the primary package, thus delivering the drug through the cannula and into the injection site. The manual injector is further configured such that, after ejection of the drug, the plunger is disengaged from the upper housing part to allow the entire primary package, together with the plunger, to be moved through the manual injector towards the upper housing part and thereby withdraw the cannula entirely into the lower housing part.

The above manual injector reduces the possibility of stick injuries and contamination/cross-infection. The known injectors do not provide a means to inform a user when the complete dose has been delivered. The user is therefore not sure when to withdraw the injector from the injection site. There is a need for an injector that indicates when a dose has been administered.

### SUMMARY

According to a first aspect of the present invention there is provided an injector for use with a primary package or syringe comprising a cannula component having a cannula, and a body containing a medicament and a bung located within the body. The injector comprises a housing configured to receive the primary package or syringe, the housing comprising upper and lower housing parts in telescopic engagement with one another. Telescopic collapsing of the parts causes the bung to be moved axially through the body of the primary package or syringe to eject medication through the cannula component. A shroud is coupled to the lower housing part and is movable between a retracted position in which the shroud does not cover a cannula tip of the cannula and an extended position in which it does cover the cannula tip. An indicating component is releasably coupled to the shroud. A biasing member is configured to bias the indicating component away from the shroud. The shroud and indicating component are configured to move together in a rearward direction through the upper housing part during said collapsing, up to a release position at which position the indicating component is released from the shroud allowing the biasing means to drive axial separation of the indicating component and the shroud. This causes the indicating component to continue rearward movement up to a stop position whilst biasing the shroud towards said extended position.

Optionally, the indicating component is releasably coupled to the shroud by a rotatable coupling. The injector may be configured to prevent relative rotation of the indicating component and the shroud during rearward movement, prior to arrival at the release position, and thereafter facilitate rotation to axially release the indicating component from the shroud.

Optionally, one or more tracks are provided in the inner surface of the upper housing part to receive anti-rotation features provided on the indicating component and the shroud to prevent relative rotation of the indicating component and the shroud. A rear end of the one or more tracks may be located substantially at the release position.

Optionally, at least an end region of the upper housing part is substantially transparent, or a window is provided in the upper housing part at the stop position.

Optionally, the stop position is defined by an upper wall of the upper housing part.

Optionally, the injector further comprises a shroud clip axially located between the shroud and the indicating component, the shroud clip being configured to move with the shroud whilst being releasably coupled to the indicating component. The shroud clip may be releasably coupled to the indicating component by rotational engagement. The rotational engagement may be facilitated by threaded engagement of the shroud clip and the indicating component.

Optionally, the biasing means is a spring coupled between the indicating component and the shroud clip.

Optionally, the shroud clip comprises a pair of axially extending legs configured to pass outside of the primary package or syringe and engage with the shroud.

Optionally, the lower housing part is configured to retain the primary package or syringe within the lower housing part against a force exerted on the primary package or syringe during telescoping together of the upper and lower housing parts.

Optionally, the injector is a manual injector.

According to a second aspect of the present invention there is provided an injection system comprising an injector assembled around a primary package or syringe.

According to a third aspect of the present invention there is provided a kit of parts for use with a primary package or syringe comprising a cannula component having a cannula and a body containing a medicament and a bung located within the body. The kit of parts comprises an upper part comprising an upper housing part, an indicating component and a biasing member; and a lower part comprising a lower housing part and a shroud. The upper housing part and the lower housing part are configured to be assembled around the primary package or syringe such that the upper housing part and lower housing part are in telescopic engagement with one another such that telescopic collapsing of the parts causes the bung to be moved axially through the body of the primary package or syringe to eject medication through the cannula component. The parts, once assembled, are configured such that the shroud is coupled to the lower housing part and movable between a retracted position in which the shroud does not cover a cannula tip of the cannula and an extended position in which it does cover the cannula tip. The parts, once assembled, are further configured such that the indicating component is releasably connectable to the shroud and the biasing member is configured to bias the indicating component away from the shroud. The parts, once assembled, are further configured such that shroud and indicating component move together in a rearward direction through the upper housing part during said collapsing, up to a release position at which position the indicating component is released from the shroud allowing the biasing means to drive axial separation of the indicating component and the shroud, causing the indicating component to continue rearward movement up to a stop position whilst biasing the shroud towards said extended position.

### BRIEF DESCRIPTION OF THE DRAWINGS

FIG. 1 is a cross-sectional view through a known primary package comprising a cartridge and a cannula component for assembly in a sterile environment;

FIG. 2 is an exploded view of a manual injector including a primary package;

FIG. 3 is a cross-sectional side view through the manual injector of FIG. 2 when assembled around the primary package of FIG. 1;

FIG. 4 is a cross-sectional side view through the manual injector of FIG. 3 with the over-cap of the lower housing part removed;

FIG. 5 is a cross-sectional side view through the manual injector of FIG. 3 with the upper housing part advanced relative to the lower housing part;

FIG. 6 is a cross-sectional side view through the manual injector of FIG. 3 with the upper housing part maximally advanced and the spring partly released;

FIG. 7 is a cross-sectional side view through the manual injector of FIG. 3 with the spring fully released and the shroud deployed; and

FIG. 8 illustrates by way of various side perspective and semi-transparent views an operating sequence within the manual injector of FIG. 3

### DETAILED DESCRIPTION

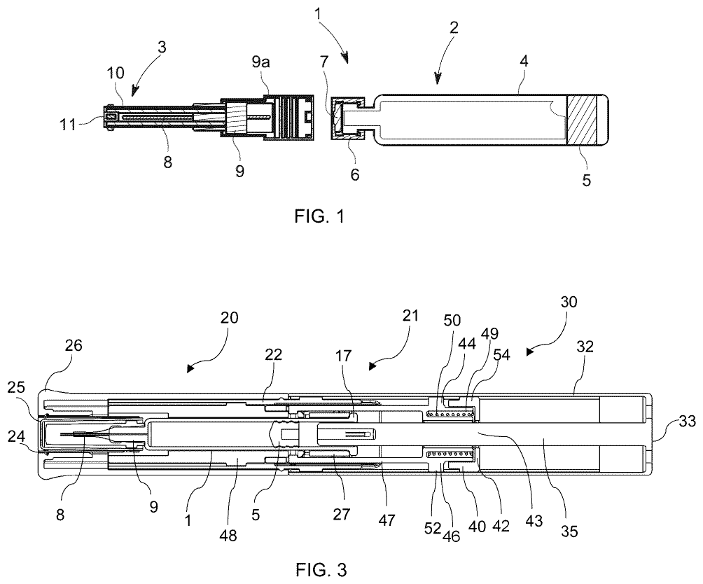

A known primary package 1 for containing a medicament or drug has been described above with reference to FIG. 1. A manual injector that facilitates an injection using such a known primary package, or indeed other primary packages or syringes, will now be described. For ease of understanding, reference will be made to upper and lower ends of the injector and the package 1, where “lower” refers to an end closest to the cannula tip and to the skin in use, whilst “upper” refers to an end furthest from the cannula tip and skin in use. It is noted that, in the context of the following discussion, the terms “needle” and “cannula” are used interchangeably and the use of one term encompasses the other, even though the precise technical meaning of each may differ.

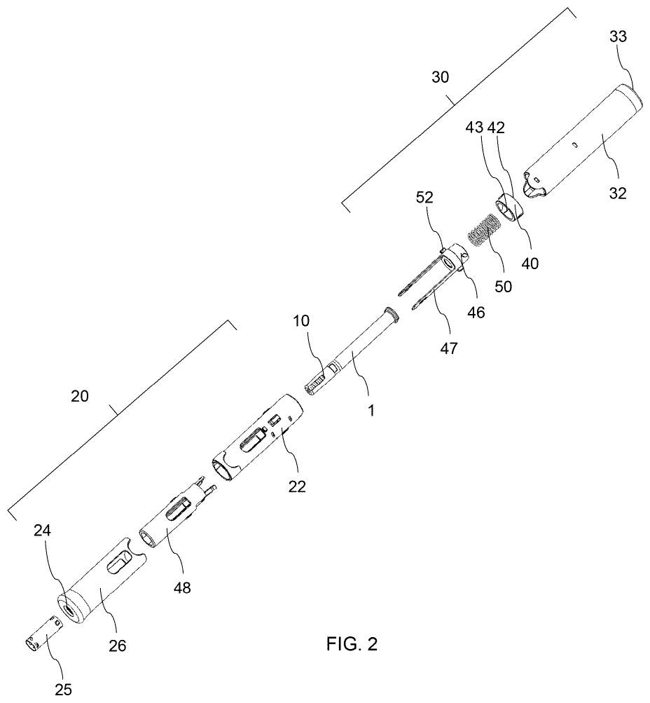

FIG. 2 illustrates an exploded view of the manual injector 21 and a primary package 1. The manual injector 21 comprises an upper part 30 configured to be arranged around a lower part 20.

The lower part 20 comprises a generally cylindrical lower housing part 22 which is generally open at its lower and upper end. With reference to FIG. 3, a pair of indented features 27 are provided on the inner surface of the lower housing part 22 to retain the primary package 1 inside the lower housing part 22. The distance between the opposing inner faces of the features 27 is marginally greater than the diameter of the cylindrical body 4 such that the body can pass between the features whilst a radially extending flange extending around the upper end of the body 4 rests on the features 27.

The lower part 20 further comprises a shroud 48 for covering the cannula component 3 of the primary package 1. The lower housing part 22 is shaped to receive and accommodate the shroud 48. The shroud 48 is slidably positioned in the lower housing part 22 and is of a generally cylindrical shape with an inner diameter large enough to surround the primary package 1 and an outer diameter marginally less than the inner diameter of the lower housing part 22. A cap 26, also of a generally cylindrical shape, is located concentrically over the lower housing part 22 and is held in place by, for example, a suitable snap fit or interference fit connection. A centrally located circular opening 24 is located in the lower end of the cap 26. A boot remover or RNS remover 25 is positioned in the opening. The lower end of the RNS remover 25 is closed so as to cover the circular opening 24.

The upper part 30 will now be described with reference FIG. 2 and FIG. 3, which illustrate a cross-sectional side view of the manual injector 21 assembled around the primary package 1. The upper part 30 comprises a generally cylindrical upper housing part 32, closed at an upper end by upper wall 33 and open at a lower end. The upper part 30 comprises a plunger 35 of generally cylindrical shape. The plunger 35 forms a fixed part of the upper part 30 and depends from the upper wall 33, extending axially through the cylindrical wall of the upper housing part 32.

The upper part 30 further comprises a “flag” 40, a shroud clip 46 and a spring which are located within the upper housing 32 and around the plunger 35. The flag 40 is slidably located within the upper housing part 32 and has a generally annular shape partially closed at an upper end by a flag wall 42. A centrally located circular opening 43 is provided in the flag wall 42 to accommodate the plunger 35. The flag 40 is positioned above the shroud clip 46 and is configured to receive and partially enclose a main body of the shroud clip 46. The main body of the shroud clip 46 has generally annular shape. The outer diameter of the flag 40 is marginally less than the inner diameter of the upper housing part 32. The inner diameter of the flag 40 is marginally greater than the outer diameter of the main body of the shroud clip 46. The inner diameter of the main body of the shroud clip 46 is marginally greater than the diameter of the plunger 35. An internal thread is provided on the inner surface of the flag 40 which complements an external projection on the outer surface of the main body of the shroud clip 46. The shroud clip 46 and flag 40 can therefore be threaded together during assembly.

The spring 50 is positioned between the flag 40 and the shroud clip 46 and received in a cylindrical recess 49 of the shroud clip 46. The opening of the cylindrical recess 49 is located concentrically on the top of the main body of the shroud clip 46 and extends radially downwards into the shroud clip 46 to receive the length of the spring 50 when it is in a compressed state.

FIG. 8, panel a), shows a perspective view of a portion of the assembled upper housing part 32. In panel a), the shroud clip 46 is shown to comprise two ribs 52 extending radially outwardly from opposing sides the main body of the shroud clip 46. The flag 40 also comprises two ribs 54 extending radially outwardly from opposing sides of the flag 40. A track or slot 56 is disposed on each side of the inner surface of the upper housing part 32 to receive respective pairs of ribs 52, 54. Upper ends of the tracks are open. The vertical distance between the upper end of the track 56 and the upper wall 33 is greater than the height of the flag 40.

Turning to FIGS. 2 and 3, the shroud clip 46 further comprises two legs 47 axially extending down the upper housing part 32 towards the lower housing part 22. The lower ends of the axially extending legs 47 are connectable to the shroud 48 by way of suitable complementary features. Whilst the spring 50 will likely, although not necessarily, be formed of a metal, other components manual injector 21 will likely be formed using plastics materials and injection moulding processes, although other materials and production processes may be used. Advantageously, the upper housing part 32 and the upper wall 33 can be formed of a transparent material to provide for observation of the flag 40. Alternatively, appropriate holes or slots may be formed in the housing to allow the flag to be viewed after firing (see below).

Assembly of the manual injector 21 firstly comprises assembling the lower and upper parts 20, 30 separately. With respect to the lower part 20, the shroud 48 is inserted into the lower housing part 22 and the cap 26 and RNS remover 25 are secured over the lower end of the lower housing part 22. With respect to the upper component 30, the spring 50 is compressed into the cylindrical recess 49 of the shroud clip 46 and the flag 40 and shroud clip 46 are threaded together until the ribs 52, 54 of the shroud clip 46 and the flag 40 align. The flag 40, shroud clip 46 and spring 50 are inserted into the upper housing part 32 around the plunger 35. The ribs 52, 54 of the shroud clip 46 and the flag 40 are aligned with and slotted into the tracks 56 in the inner surface of the upper housing part 32 to prevent relative rotation of the flag 40 and the shroud clip 46. This maintains the spring 50 in a compressed state. The primary package 1 is then inserted into the lower part 20 until the upper end 17 of the primary package 1 contacts the features 27 of the lower housing part 22. The primary package 1 is pressed in so that features on the end of the RNS 10 engage with complimentary features of the RNS remover 25 in a known manner. The upper and lower parts 20, 30 are aligned relative to each other such that the legs 47 of the shroud clip 46 are out of alignment with the features 27 of the lower housing part 22. The open end of the upper part 30 is then positioned over and around the end of the lower part 20 such that a lower end of the plunger 35 abuts the moveable bung 5 within the cartridge body 4. In this position, complimentary features contained within the lower and upper parts 20, 30, such as the shroud 48 and the shroud clip 46, as well as features provided on the lower and upper housing parts 22, 32, are engaged to each other to fix the lower and upper parts 20, 30 together. These complimentary features are likely snap-fit features although other forms of engagement may be contemplated, e.g. screw threads.

As will be apparent from FIG. 3, in this assembled, pre-use state, little or no force is applied between the cartridge 2 and the cannula component hub 9 (or cannula 8), such that the upper end of the cannula 8 remains spaced apart from the sealing element 7. The spring 50 is compressed between the shroud clip 46 and the flag 40 and abuts the lower side of the flag wall 42. In this assembled, pre-use state, the insertion of the respective ribs 52, 54 of the shroud clip 46 and the flag 40 prevents the flag 40 rotating relative to the shroud clip 46, in turn maintaining the spring 50 in a compressed state. Features of the lower and upper housing parts 22, 32 e.g. the same features fixing the housing parts together, provide a resistance to further movement of the lower housing part 22 into the upper housing part 32 which must be overcome to commence injection. Furthermore, any such movement is prevented by the cap 26 which is in blocking engagement with the upper housing part 32. The configuration of the injector illustrated in FIG. 3 is typically that in which the injector is provided to an end user or healthcare professional.

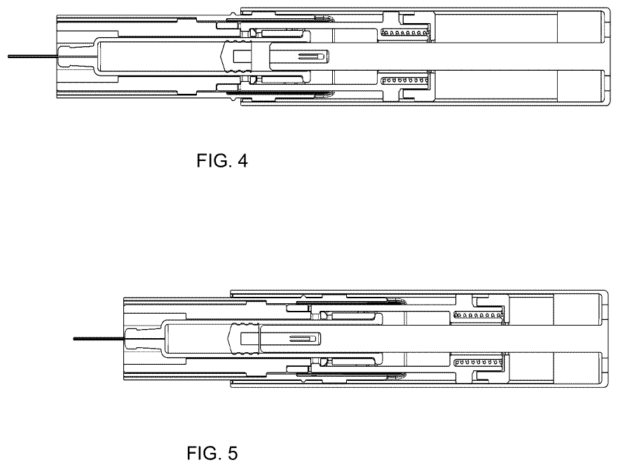

FIG. 4 illustrates a first step in the operation of the manual injector 21 and in which the cap 26 and the RNS remover 25 have been pulled off of the lower housing part 22. This action also pulls the RNS 10, which is captured by the RNS remover 25, off of the cannula component 3, thereby exposing a lower end of the cannula 8 outside of the lower housing part 22. This breaks the sterile barrier. A latch between the hub 9 and the body 9a of the cannula component, or a simple “bump”, is provided to prevent the RNS removal step from pulling the hub 9 forward and potentially damaging the cannula 8 or bringing it into contact with the sealing element 7.

An injection can then be performed. This typically involves the user grasping the upper upper housing part 32 within his or her fist, and pressing the lower end of the manual injector 21 against the injection site. This is typically done in a stabbing motion such that cannula penetration and injection occurs in a quick and continuous manner. For the purpose of explanation, FIGS. 5 to 7 illustrate various stages in this injection process.

FIG. 5 illustrates a stage at which the cannula 8 is penetrating the skin at the injection site and force is applied between the lower and upper housing parts 22, 32 to cause the upper housing part 32 and plunger 35 to advance into the lower housing part 22, applying force to the bung 5 as it does so. As the user continues to apply force between the upper housing part 32 and the skin, the hub 9, and with it the cannula 8, are pushed back through the cylindrical body 9a so that the upper tip of the cannula passes through the sealing element 7, bringing the flow channel of the cannula into fluid communication with the drug. Further force applied by the user will now advance the plunger 35 and bung 5 through the cartridge body 4 of the primary package 1, thus delivering the drug through the cannula 8 and into the injection site.

It will be appreciated from FIG. 8, panel a) that the upper housing part 32 will advance relative to the lower housing part 22 until the lower housing 22 comes into contact with the shroud clip 46. As the lower and upper housing parts continue to advance, the shroud clip 46 and flag 40 will be pushed along the track 56.

Drug delivery is completed when the lower housing part 22 has advanced into the upper housing part 32 by some predetermined distance. As illustrated in FIG. 8, panel b), once the lower housing part 22 has advanced by the predetermined distance, the ribs 52 of the flag 40 exit from the respective tracks 56, whist the ribs 54 of the shroud clip are still prevented from rotating. The relative force exerted between the flag and the shroud clip causes the flag 40 to rotate relative to the shroud clip 46. The thread by which the flag 40 is attached to the shroud clip 46 is relatively short such that, very quickly, the flag is completely unwound and freed from the shroud clip 46.

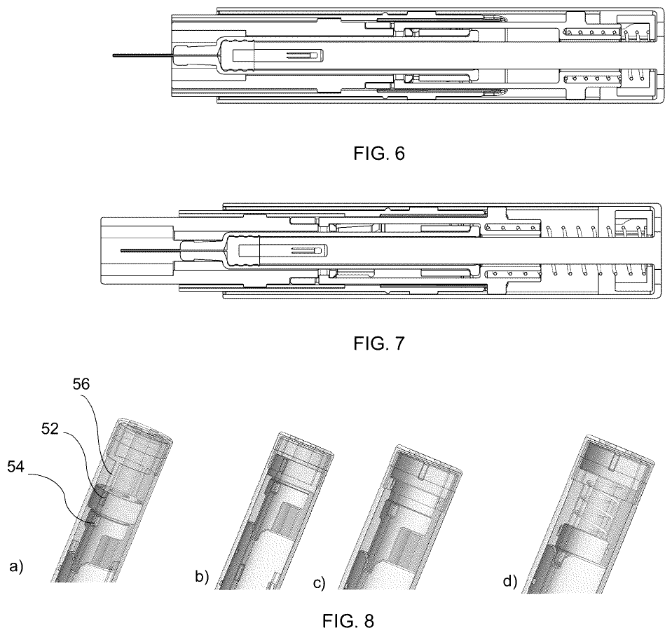

As illustrated in and FIG. 6 and FIG. 8, panel c), as the spring 50 continues to expand, the flag 40 is driven upwards to hit the upper wall 33. This produces an audible “clicking” noise to alert the user that the dose has been delivered. Moreover, the displacement of the flag 40 is visible through the upper housing part 32 and the upper wall 33. This provides the user with a visual indication that the dose has been delivered. At this point, the user will remove the manual injector 21 away from the injection site.

With the lower housing part 22 no longer pressing against the upper housing part 32, the remainder of the tension in the spring 50 is released. As a result, the shroud 48 is deployed. This is illustrated in FIG. 7 and FIG. 8, panel d). With the spring in the fully extended state, the shroud clip 46 and shroud 48 are deployed, whereby the shroud clip 46 and shroud 48 are displaced downwards and the lower part of the shroud 48 extends beyond the lower housing part 22 to cover the exposed cannula 8. The possibility of stick injuries and contamination/cross-infection is eliminated or at least greatly reduced.

The mechanism described with respect to the accompanying figures provides a very simple and elegant solution to the need to indicate when a dose has been delivered and subsequently shield a cannula following withdrawal of the injector from the injection site. The solution involves releasing a flag within the device once drug delivery is complete, and subsequently deploying a shroud from within the device once the injector has been removed.

Whilst the solution is well suited for use with a manual injector, the mechanism might also be usefully incorporated into an autoinjector which incorporates some force delivery mechanism, such as a spring, motor or pneumatic source, to push the plunger through the device.

It will be appreciated by the skilled person that various modifications may be made to the above described embodiments without departing from the scope of the present invention. For example, in certain embodiments the flag or indicating component may be connected directly to the shroud, avoiding the need for a shroud clip.

1. An injector for use with a primary package or syringe comprising a cannula component having a cannula, and a body containing a medicament and a bung located within the body, the injector comprising:
a housing configured to receive the primary package or syringe, the housing comprising upper and lower housing parts in telescopic engagement with one another such that telescopic collapsing of the parts causes the bung to be moved axially through the body of the primary package or syringe to eject medication through the cannula component;
a shroud coupled to the lower housing part and movable between a retracted position in which the shroud does not cover a cannula tip of the cannula and an extended position in which it does cover the cannula tip;
an indicating component releasably coupled to the shroud;
a biasing member configured to bias the indicating component away from the shroud;
wherein the shroud and indicating component are configured to move together in a rearward direction through the upper housing part during said collapsing, up to a release position at which position the indicating component is released from the shroud allowing the biasing means to drive axial separation of the indicating component and the shroud, causing the indicating component to continue rearward movement up to a stop position whilst biasing the shroud towards said extended position.

2. An injector according to claim 1, wherein the indicating component is releasably coupled to the shroud by a rotatable coupling, the injector being configured to prevent relative rotation of the indicating component and the shroud during rearward movement, prior to arrival at the release position, and thereafter facilitate rotation to axially release the indicating component from the shroud.

3. An injector according to claim 2, wherein one or more tracks is provided in the inner surface of the upper housing part to receive anti-rotation features provided on the indicating component and the shroud to prevent relative rotation of the indicating component and the shroud, and wherein a rear end of the one or more tracks is located substantially at the release position.

4. An injector according to claim 1, wherein at least an end region of the upper housing part is substantially transparent, or wherein a window is provided in the upper housing part at the stop position.

5. An injector according to claim 1, wherein the stop position is defined by an upper wall of the upper housing part.

6. An injector according to claim 1, further comprising a shroud clip axially located between the shroud and the indicating component, the shroud clip being configured to move with the shroud whilst being releasably coupled to the indicating component.

7. An injector according to claim 6, wherein the shroud clip is releasably coupled to the indicating component by rotational engagement.

8. An injector according to claim 7, wherein said rotational engagement is facilitated by threaded engagement of the shroud clip and the indicating component.

9. An injector according to claim 6, wherein the biasing means is a spring coupled between the indicating component and the shroud clip.

10. An injector according to claim 6, wherein the shroud clip comprises a pair of axially extending legs configured to pass outside of the primary package or syringe and engage with the shroud.

11. An injector according to claim 1, the lower housing part being configured to retain the primary package or syringe within the lower housing part against a force exerted on the primary package or syringe during telescoping together of the upper and lower housing parts.

12. An injector according to claim 1, wherein the injector is a manual injector.

13. An injection system comprising the injector according to claim 1 assembled around a primary package or syringe.

14. A kit of parts for use with a primary package or syringe comprising a cannula component having a cannula and a body containing a medicament and a bung located within the body, the kit of parts comprising:
an upper part comprising an upper housing part, an indicating component and a biasing member; and
a lower part comprising a lower housing part and a shroud,
wherein the upper housing part and the lower housing part are configured to be assembled around the primary package or syringe such that the upper housing part and lower housing part are in telescopic engagement with one another such that telescopic collapsing of the parts causes the bung to be moved axially through the body of the primary package or syringe to eject medication through the cannula component, the parts, once assembled, being configured such that:
the shroud is coupled to the lower housing part and movable between a retracted position in which the shroud does not cover a cannula tip of the cannula and an extended position in which it does cover the cannula tip;
the indicating component is releasably connectable to the shroud; and
the biasing member is configured to bias the indicating component away from the shroud; and
the shroud and indicating component are configured to move together in a rearward direction through the upper housing part during said collapsing, up to a release position at which position the indicating component is released from the shroud allowing the biasing means to drive axial separation of the indicating component and the shroud, causing the indicating component to continue rearward movement up to a stop position whilst biasing the shroud towards said extended position.
